(19) United States
(12) Patent Application Publication
(10) Pub. No.: US 2017/0181694 A1
(43) Pub. Date: Jun. 29, 2017

(54) SYSTEM AND METHOD FOR PROVIDING TRANSITIONAL MONITORING ALONG SEDATION CONTINUUM

(71) Applicant: Ethicon Endo Surgery, Inc., Cincinnati, OH (US)

(72) Inventors: Paul J. Niklewski, Cincinnati, OH (US); James F. Martin, Lebanon, OH (US)

(21) Appl. No.: 14/982,210

(22) Filed: Dec. 29, 2015

(51) Int. Cl. A61B 5/00; A61B 5/0205; A61M 16/06; A61M 5/172; A61M 16/10; A61B 5/1455; A61M 5/142

(52) U.S. Cl. CPC A61B 5/4839; A61B 5/14551; A61B 5/0205; A61B 5/4064; A61B 5/742; A61B 5/7405; A61B 5/4821; A61M 5/142; A61M 5/1723; A61M 16/1005; A61B 5/7278; A61M 16/0666; A61B 5/021

(57) ABSTRACT

A transition monitor receives data from a variety of sensors that provide data indicative of the level of sedation of a patient, such as a bispectral index monitor, automated responsiveness monitor, pulse oximeter, or other device. As data is received from a sensor device, the transition monitor will apply a weighting calculation and algorithm to the received value in order to determine a weighted value for the received data. The weighted value is used, along with prior values, to determine a sedated patient's position within a continuum of sedation levels, such as minimal sedation, moderate sedation, or deep sedation. When a newly received value triggers a transition from one sedation level to a next sedation level, one or more thresholds, safeguards, or drug delivery configurations may be modified to adapt to the change within the sedation continuum.

### BACKGROUND

Patient monitoring systems may be used to monitor physiological parameters of patients undergoing diagnostic procedures, surgical procedures, and/or various other types of medical procedures. In some settings, a nurse or technician in a pre-procedure room may prepare a patient for an upcoming procedure. This preparation may include connecting monitors to the patient for the purpose of obtaining baseline data to be used in the procedure. Such monitors may include a blood pressure monitor and pulse oximetry monitor, among others. Blood pressure readings may be taken by a blood pressure cuff, whereby a nurse or technician secures the cuff around a patient's arm and uses a device to pump air into the cuff Once the reading from the cuff stabilizes, the nurse or technician may have to manually record the data (e.g., handwritten on a sheet of paper or typed into a portable electronic device), and save this information for later reference during the procedure and eventually, for a patient report. For the nurse or technician to take a pulse oximeter reading, he or she may have to boot up the pulse oximeter module, secure a pulse oximeter probe upon the patient, and take a reading of the patient. This reading may also be written down on paper or otherwise be manually recorded for later use. Once it is determined the patient is ready for the procedure, the nurse or technician may have to disengage the blood pressure cuff and pulse oximetry probes from the patient, so the patient can be transported from the pre-procedure room to the procedure room.

After the patient enters the procedure room and before the procedure begins, several tasks may be needed to prepare the patient for the procedure. The nurse or technician may have to reconnect both blood pressure and pulse oximetry readers before the procedure can begin. In addition to blood pressure and pulse oximetry, other connections such as, for example, capnography, supplemental oxygen, and electrocardiogram may be required. A great deal of time may be required to connect the physiological monitors to the patient and to connect the physiological monitors to the monitoring system. In some such instances, the nurse or technician must spend time reconnecting the same kinds of physiological monitors that were previously connected to the patient in the pre-procedure room. The time it takes to make these connections may occupy valuable procedure room time, thus decreasing practice efficiency.

In various settings, it may also be desirable to deliver drugs to a patient during a procedure, such as via an IV and/or face mask, etc. Such drugs may include sedatives, anelgesics, amnestics, etc. In some instances, such drugs may be selected and/or combined to place a patient in a state of “conscious sedation” (in lieu of simply rendering a patient completely unconscious through a general anesthetic). Certain systems may also be used to automate the delivery of such drugs. For instance, such systems may be located in the same room where a medical procedure is performed, and may be coupled with a physiological monitoring system to automatically tailor the delivery of drugs based on patient parameters detected by the monitoring system. Examples of such systems are disclosed in U.S. Pat. No. 6,745,764, entitled “Apparatus and Method for Providing a Conscious Patient Relief from Pain and Anxiety Associated with Medical or Surgical Procedures,” issued Jun. 8, 2004, the disclosure of which is incorporated by reference herein; U.S. Pat. No. 7,833,213, entitled “Patient Monitoring and Drug Delivery System and Method,” issued Nov. 16, 2010, the disclosure of which is incorporated by reference herein; U.S. Pat. No. 7,935,081, entitled “Drug Delivery Cassette and a Medical Effector System,” issued May 3, 2011, the disclosure of which is incorporated by reference herein; U.S. Pub. No. 2009/0292179, entitled “Medical System having a Medical Unit and a Display Monitor,” published Nov. 26, 2009, the disclosure of which is incorporated by reference herein; and U.S. Pub. No. 2010/0010433, entitled “Medical System which Controls Delivery of a Drug,” published Jan. 14, 2010, the disclosure of which is incorporated by reference herein.

One difficulty that may be encountered with some drug delivery and monitoring systems is that individual monitors may not be consistently precisely indicative of a response to drug delivery, depending upon the patient's current condition. For example, an automated responsiveness monitor (“ARM”) may provide data that is a generally reliable indicator of a minimal sedation level for a patient undergoing a medical procedure under sedation. However, once the patient transitions beyond a moderate level of sedation the ARM may be providing no data whatsoever, and may thus not be helpful in determining transitions between deep sedation and moderate sedation. Similarly, a bispectral index (“BIS”) monitor may provide a reliable indicator of consciousness within deep and moderate sedation. However, due to increased brain activity during minimal sedation, a BIS may provide a flood of unhelpful or deceptive feedback. This may create problems for clinicians who must rely on these devices during a procedure, as it can be difficult to determine whether data coming from a particular monitor is reliable, especially during a sedation procedure where transitions from one level of sedation to another may not be immediately apparent.

While a variety of systems have been made and used for monitoring patients and delivering drugs to patients, it is believed that no one prior to the inventor(s) has made or used the technology as described herein.

### BRIEF DESCRIPTION OF THE DRAWINGS

It is believed the present invention will be better understood from the following description of certain examples taken in conjunction with the accompanying drawings, in which like reference numerals identify the same elements and in which:

FIG. 1 depicts a perspective view of an exemplary patient monitoring and drug delivery system;

FIG. 2 depicts a perspective view of the patient monitoring unit of the system of FIG. 1;

FIG. 3 depicts a perspective view of the drug delivery unit of the system of FIG. 1;

FIG. 4 depicts a block diagrammatic view of the system of FIG. 1 with additional exemplary components;

FIG. 5 depicts a schematic view of an exemplary set of devices providing data to a monitoring device such as the patient monitoring unit of FIG. 2;

FIG. 6 depicts a flowchart of exemplary steps that may be performed to maintain a sedation continuum over a period of time using the system of FIG. 5;

FIG. 7 depicts a flowchart of exemplary steps that may be performed to apply varying weights to data received from a sensor of the system of FIG. 5;

FIG. 8 depicts a flowchart of exemplary steps that may be performed to recalculate and maintain a sedation continuum in response to data received from a sensor of the system of FIG. 5; and

FIG. 9 depicts a flowchart of exemplary steps that may be performed to transition drug delivery configurations from a current stage of a sedation continuum to a subsequent stage of a sedation continuum using the system of FIG. 5.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the technology should not be used to limit its scope. Other examples, features, aspects, embodiments, and advantages of the technology will become apparent to those skilled in the art from the following description, which is by way of illustration, one of the best modes contemplated for carrying out the technology. As will be realized, the technology described herein is capable of other different and obvious aspects, all without departing from the technology. Accordingly, the drawings and descriptions should be regarded as illustrative in nature and not restrictive.

It is further understood that any one or more of the teachings, expressions, embodiments, examples, etc. described herein may be combined with any one or more of the other teachings, expressions, embodiments, examples, etc. that are described herein. The following-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

I. Exemplary Conscious Sedation System

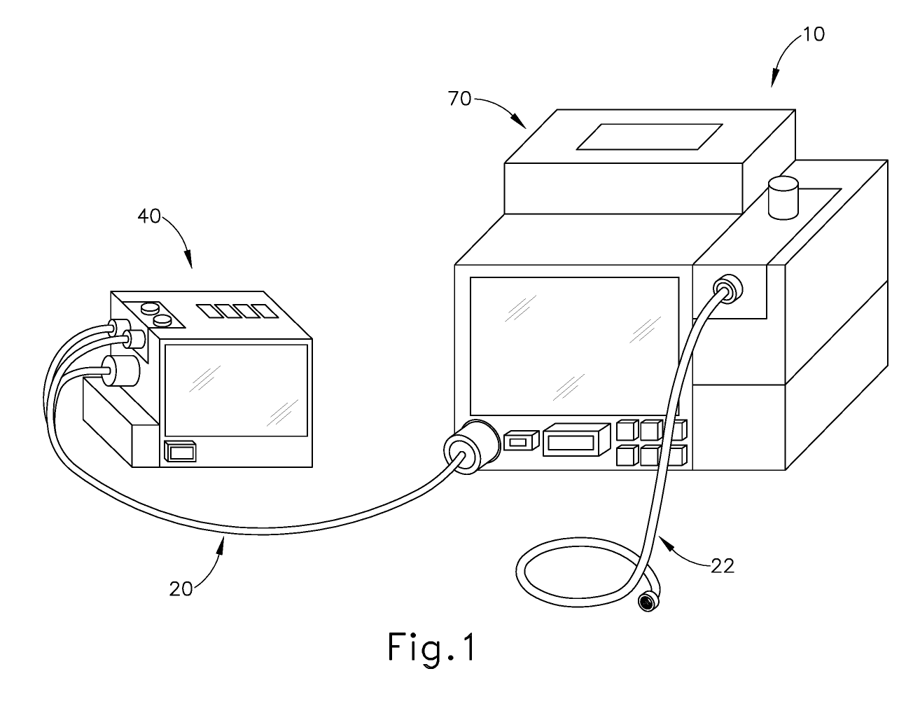

FIG. 1 shows an exemplary patient care system (10) comprising a bedside monitor unit (BMU) (40) and a procedure room unit (PRU) (70). One exemplary use of patient care system (10) is to monitor patient parameters and deliver sedative, analgesic, and/or amnestic drugs to a conscious, non-intubated, spontaneously-ventilating patient undergoing a diagnostic procedure, surgical procedure, or other medical procedure by a physician. This use is not exhaustive of all of the potential uses of the invention but will be used to describe examples herein. BMU (40) and PRU (70) are connected via communication cable (20). Communication cable (20) provides means for transmitting electronic data as well as various hydraulic signals and gases between BMU (40) and PRU (70). For instance, communication cable (20) may include a plurality of pneumatic tubes and a plurality of electrical wires, all integrated within a single sheath or cable.

Communication cable (20) may be removed from both BMU (40) and PRU (70) to facilitate practice efficiency and user convenience. BMU (40) and PRU (70) are free to move independently of each other if communication cable (20) is not in place. This allows for mobility of each unit independent of the other; this feature is especially important in hospitals that have a great deal of medical procedures and there is little time to connect patients to monitors. BMU (40) and PRU (70) preferably accommodate an external oxygen source that is intended to provide supplemental oxygen to the patient during the course of a surgical procedure if the clinician so desires. An IV tube set (22) is shown connected to PRU (70) and delivers sedative or amnestic drugs to a patient during a surgical procedure.

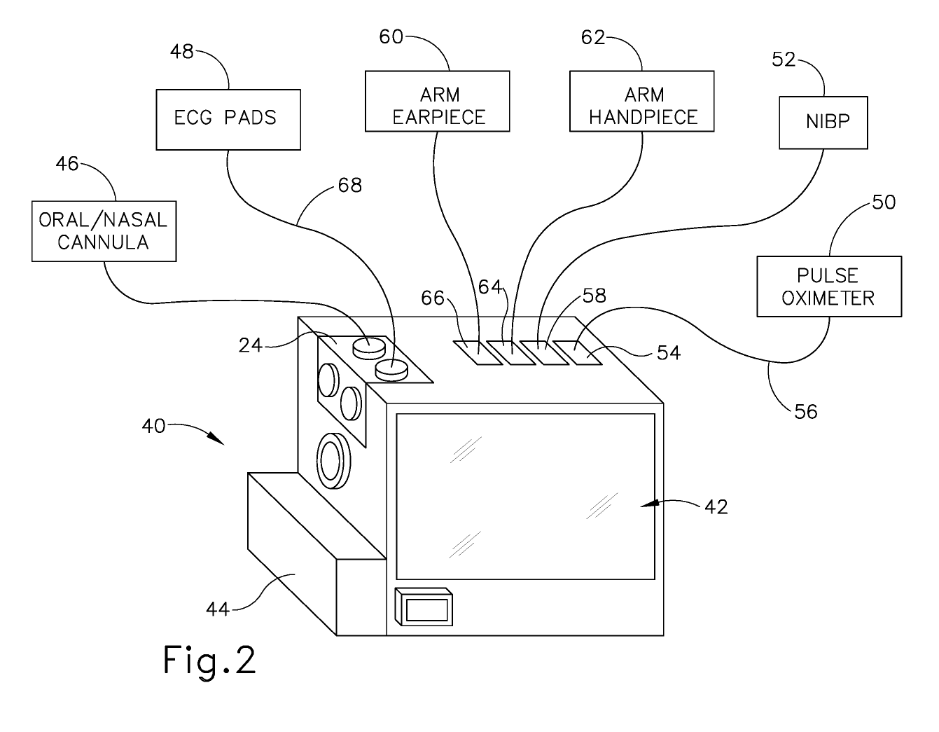

BMU (40) serves as a patient monitoring unit, monitoring various physiological parameters of a patient. As shown in FIG. 2, BMU (40) is compact and portable so it requires relatively little effort to move from one room to another. In some versions, BMU (40) could mount upon either an IV pole or a bedrail; this would free the clinician from the burden of carrying the unit wherever the patient needs to be transported. BMU (40) is small and light enough to be held in the hand of a nurse or technician. BMU (40) allows the user to input information via a touch screen assembly (42) or a simple keypad, etc. Touch screen assembly (42) is provided as an overlay on a display device that is integrated into one surface of BMU (40), and that displays patient and system parameters, and operational status of BMU (40). An exemplary bedside touch screen assembly (42) is a 5.25″ resistive touch screen manufactured by MicroTech mounted upon a 5.25″ color LCD screen manufactured by Samsung. Other suitable forms that a display screen and touch screen may take will be apparent to those of ordinary skill in the art in view of the teachings herein. An attending nurse or physician may enter patient information such as, for example, patient weight and a drug dose profile into BMU (40) by means of bedside touch screen assembly (42). A BMU battery (44) is fixedly attached to the BMU (40) and comprises a standard rechargeable battery such as, for example, Panasonic model no. LC-T122PU, that is capable of supplying sufficient power to run BMU (40) for an extended period of time. In some versions, BMU battery (44) can be recharged while BMU (40) is connected to PRU (70) via communication cable (20) or can be charged directly from an independent power source. Various suitable ways in which battery (44) may be charged will be described in greater detail below in section III.A.; while still other suitable ways will be apparent to those of ordinary skill in the art in view of the teachings herein. Similarly, various suitable forms that battery (44) may take, as well as various suitable compositions thereof, will be apparent to those of ordinary skill in the art in view of the teachings herein.

As shown in FIG. 2, BMU (40) may be connected to a plurality of patient sensors and peripherals used to monitor patient vital signs and deliver supplemental oxygen to the patient. Oral nasal cannula (46) delivers oxygen from an external oxygen source and collects samples of exhaled gas. Oral nasal cannula (46) is removably attached to cable pass-through connection (24). Cable pass-through connection (24) sends the signal obtained by oral nasal cannula (46) directly to a capnometer (e.g., a CardioPulmonary Technologies CO2WFA OEM) in PRU (70) and preferably via communication cable (20) (FIG. 1). The capnometer measures the carbon dioxide levels in a patient's inhalation/exhalation stream via a carbon dioxide-sensor as well as measuring respiration rate. Also attached to the cable pass-through connection (24) is a standard electrocardiogram (ECG) (48), which monitors the electrical activity in a patient's cardiac cycle. The ECG signals are sent to the PRU (70) where the signals are processed. A pulse oximeter probe (50) (e.g., by Dolphin Medical) and a non-invasive blood pressure (NIBP) cuff (52) are also connected to BMU (40) in the present example. Pulse oximeter probe (50) measures a patient's arterial saturation and heart rate via an infrared diffusion sensor. The data retrieved by pulse oximeter probe (50) is relayed to pulse oximeter module (54) (e.g., by Dolphin Medical) by means of pulse oximeter cable (56). The NIBP cuff (52) (e.g., a SunTech Medical Instruments PN 92-0011-00) measures a patient's systolic, diastolic, and mean arterial blood pressure by means of an inflatable cuff and air pump (e.g., by SunTech Medical), also incorporated as needed. NIBP cuff (52) is removably attached to NIBP module (58) located on BMU (40).

In the present example, a patient's level of consciousness is detected by means of an Automated Responsiveness Measurement System (ARM), though like various other components described herein, an ARM system is merely optional and is not required. An exemplary ARM system is disclosed in U.S. Pub. No. 2005/0070823, entitled “Response Testing for Conscious Sedation Involving Hand Grip Dynamics,” published Mar. 31, 2005, the disclosure of which is incorporated by reference herein. The ARM system of the present example comprises a query initiate device and a query response device. The ARM system operates by obtaining the patient's attention with the query initiate device and commanding the patient to activate the query response device. The query initiate device may comprise any type of stimulus device such as a speaker via an earpiece (60), which provides an auditory command to a patient to activate the query response device. The query response device of the present example comprises is a handpiece (62) that can take the form of, for example, a toggle or rocker switch or a depressible button or other moveable member hand held or otherwise accessible to the patient so that the member can be moved or depressed by the patient upon the patient's receiving of the auditory signal or other instruction to respond. Alternatively, a vibrating mechanism may be incorporated into handpiece (62) that cues the patient to activate the query response device. For instance, in some versions, the query initiate device comprises a cylindrical handheld device (62), containing a small 12V DC bi-directional motor enabling the handheld device to vibrate the patient's hand to solicit a response.

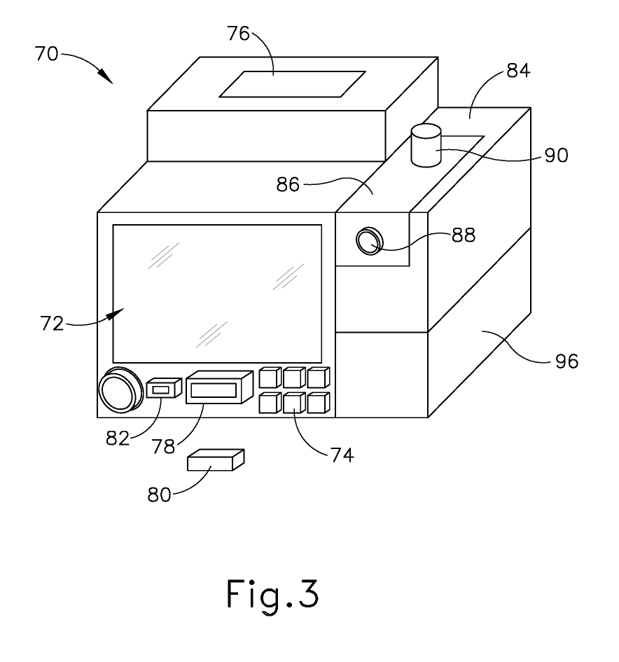

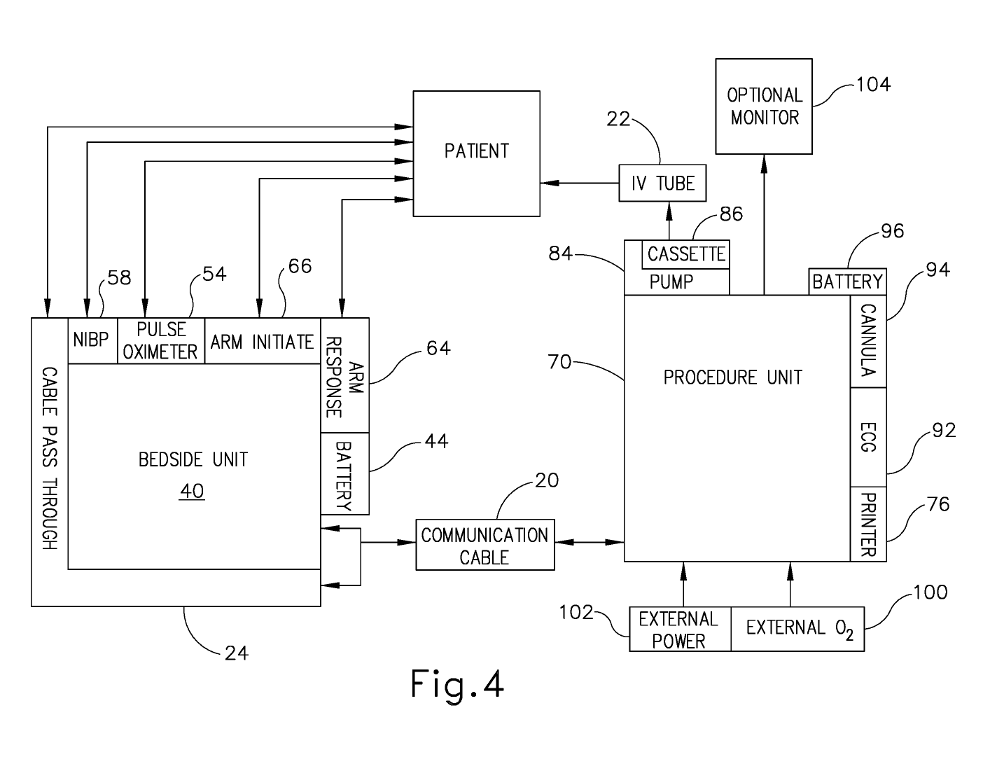

After the query is initiated, the ARM system generates signals to reflect the amount of time it took for the patient to activate the query response device in response to the query initiate device. These signals are processed by a logic board located inside BMU (40) and are displayed upon either bedside touch screen assembly (42), procedure touch screen assembly (72) (FIG. 3), and/or an optional monitor 104 (FIG. 4). The amount of time needed for the patient to respond to the query gives the clinician an idea as to the sedation level of the patient. The ARM system has two modules in this example, including a query response module (64) and a query initiate module (66), collectively referred to as the ART system modules (64, 66). ARM system modules (64, 66) have all the necessary hardware to operate and connect the query response device (62) and the query initiate device (60) to BMU (40).

In some versions monitoring modules (54, 58, 64, 66) are easily replaceable with other monitoring modules in the event of malfunction or technological advancement. These modules (54, 58, 64, 66) include all of the necessary hardware to operate their respective peripherals. The above-mentioned patient modules (54, 58, 64, 66) are connected to a microprocessor-based electronic controller or computer (MLB) located within each of the PRU (70) and BMU (40). The electronic controller or main logic board comprises a combination of available programmable-type microprocessors and other “chips,” memory devices and logic devices on various board(s) such as, for example, those manufactured by Texas Instruments (e.g., XK21E) and National Semiconductor (e.g., HKL72), among others. Various other suitable forms that modules (54, 58, 64, 66) and associated electronics may take will be apparent to those of ordinary skill in the art in view of the teachings herein.

Once BMU (40) and PRU (70) are connected via communication cable (20), ECG and capnography may be monitored, and supplemental oxygen may be delivered to the patient. It should be understood, however, that these connections may be made in the pre-procedure room to increase practice efficiency. By making these connections in the pre-procedure room, less time may be required in the procedure room connecting capnography, ECG and supplemental oxygen to PRU (70). Oral nasal cannula (46) and ECG leads (68) are connected directly to cable pass-through connection (24). Cable pass-through connection (24), located on BMU (40), is essentially an extension of communication cable (20), which allows the signals from ECG leads (68) and oral nasal cannula (46) to bypass BMU (40) and be transferred directly to PRU (70). It will be evident to those skilled in the art, however, that the BMU (40) could be configured to accept the ECG (48) and oral/nasal cannula (46) signals and process the signals accordingly to provide the information on screen (42) and supplemental oxygen to the patient in the pre-procedure room. Other examples of components, features, and functionality that may be incorporated into BMU (40) will be described in greater detail below; while still further examples of components, features, and functionality that may be incorporated into BMU (40) will be apparent to those of ordinary skill in the art in view of the teachings herein.

Referring now to FIG. 3, PRU (70) allows a physician to safely deliver drugs, such as sedative, analgesic, and/or amnestic drugs to a patient, and monitor the patient during a medical procedure. Procedure touch screen assembly (72) comprises a display device that is integrated into the surface of PRU (70), which displays patient and system parameters, and operation status of PRU (70). In some versions, procedure touch screen assembly (72) comprises a 15″ resistive touch screen manufactured by MicroTech mounted upon a 15″ color LCD screen manufactured by Samsung. Other suitable forms that a display screen and touch screen may take will be apparent to those of ordinary skill in the art in view of the teachings herein. It should be noted that, in the present example, procedure touch screen assembly (72) is the primary display and user input means, and is significantly larger than the bedside touch screen assembly (42) and is capable of displaying more detailed information. In addition to procedure touch screen assembly (72), the user may input information into PRU (70) by means of drug delivery controls (74). Drug delivery controls (74), such as buttons, dials, etc., are located on one side of PRU (70) and allow the clinician to change various system parameters and bypass procedure touch screen assembly (72). A printer (76) is integrally attached to the top of PRU (70). Printer (76) allows the clinician to print a patient report that includes patient data for pre-op and the procedure itself. The combination of printing a patient report and the automatic data logging features may decrease the amount of time and effort a nurse or technician must spend regarding patient condition during the course of a procedure. Printer (76) receives data signals from a printer interface (e.g., Parallel Systems CK205HS), which is located on the main logic board. Printer (76) may comprise a thermal printer (e.g., Advanced Printing Systems (APS) ELM 205HS) and/or any other suitable type of printer. It should also be understood that printer (76) may be remote from PRU (70) and may even be omitted altogether, if desired.

Memory card reader (78), which includes a slot in the outer casing of PRU (70), allows flash memory card (80) to be inserted and removed from PRU (70). Flash memory card (80) is a solid-state storage device used for easy and fast information storage of the data log generated by PRU (70). The data is stored so that it may be retrieved from flash memory card (80) at a later time. In some versions, memory card reader (78) accepts flash memory card (80) containing software to upgrade the functionality of patient care system (10). Again, as with other components described herein, memory card reader (78) may be modified, substituted, supplemented, or omitted as desired. In the present example, memory card reader (78) is supplemented with a data port (82). Data port (82) may include, but is not limited to, a standard serial port, a USB port, a RS232 port, an Ethernet port, or a wireless adapter (e.g., using IEEE 802.11n/g/b/a standard, etc.). Data port (82) may be used to link PRU (70) to an external printer to print a patient report or to transfer electronic files to a personal computer or mainframe. A merely illustrative example of how data port (82) may be used to communicate with a centralized network system component will be described in greater detail below in section III. B., while still other suitable examples will be apparent to those of ordinary skill in the art in view of the teachings herein.

PRU (70) delivers fluid to a patient via an infusion pump, such as a peristaltic infusion pump (84) (e.g., by B-Braun McGaw). Peristaltic infusion pump (84) is integrally attached to PRU (70), and uses peristaltic fingers to create a wavelike motion to induce fluid flow inside a flexible tube connected to a fluid reservoir. A drug cassette (86) is a generally rectangular shaped structure that is placed adjacent to peristaltic infusion pump (84). Drug cassette (86) of this example is made of a rigid thermoplastic such as, for example, polycarbonate. Drug cassette (86) has an internal cavity that houses IV tubing (22) made of a flexible thermoplastic such as, for example, polypropylene (e.g., Kelcourt). Drug cassette (86) receives tubing (22) via a port (88) and accurately and reliably positions exposed IV tubing (22) in contact with the peristaltic fingers of peristaltic infusion pump (84). IV tube set (22) attaches to a fluid vial (90), and a portion of the length of IV tube set (22) is contained within drug cassette (86). Another portion of IV tube set (22) lies external to drug cassette (86) to facilitate the interaction with peristaltic pump (84). IV tubing (22) is coiled within drug cassette (86) and has a length to reach a patient removed from the PRU (70). A fluid detection sensor (not shown) may be mounted to an inner wall of drug cassette (86). Such a fluid detection sensor may comprise any one of known fluid sensors, such as the MTI-2000 Fotonic Sensor, or the Microtrak-II CCD Laser Triangulation Sensor both by MTI Instruments Inc. IV tube set (22) may run through the fluid detection sensor before exiting drug cassette (86). PRU (70) may include features operable to prime IV tubing (22) with relative ease for a user. Various examples of how such priming may be provided are disclosed in U.S. Pat. No. 7,833,213, the disclosure of which is incorporated by reference herein.

In the present example, drug cassette (86) includes just one vial (90). However, it should be understood that some versions of drug cassette (86) may include several vials (90). Such vials (90) may include the same drug. Alternatively, a plurality of vials (90) associated with a single drug cassette (86) may include a variety of different kinds of drugs. In other words, a single drug cassette (86) may be used to selectively deliver two or more drugs simultaneously and/or in a particular sequence. While vials (90) are used in the present example, it should be understood that any other suitable type of container may be used as will be understood by those of ordinary skill in the art in view of the teachings herein. It should also be understood that some versions of PRU (70) may be configured to receive two or more drug cassettes (86). Each such drug cassette (86) may be associated with a single drug (e.g., different drug cassettes (86) used for different drugs), or each drug cassette (86) may be associated with a combination of drugs (e.g., different drug cassettes (86) used for different combinations of drugs).

FIG. 4 shows how components of system (10) interface with each other and with a patient. While not shown in FIG. 3, FIG. 4 shows how PRU (70) includes an integral ECG module (92) and integral cannula module (94). ECG module (92) is coupled with ECG (48) via ECG leads (68) extending from pass-through connection (24). Cannula module (94) is coupled with oral/nasal cannula (46), also through pass-through connection (24). Like modules (54, 58, 64, 66) described above, modules (92, 94) may be easily replaceable with other monitoring modules in the event of malfunction or technological advancement. Modules (92, 94) may also include all of the necessary hardware to operate their respective peripherals, and may be further coupled with a microprocessor-based electronic controller or computer located within PRU (70) and/or BMU (40).

As also shown in FIG. 4, PRU (70) of the present example is coupled with an external oxygen source (100), an external power source (102), and an external monitor (104). External oxygen source (100) may by regulated by one or more components of PRU (70), which may deliver oxygen from oxygen source (100) to the patient based on one or more parameters sensed by BMU (40), based on drug delivery from cassette (86), and/or based on other factors. External power source (102) may be used as a primary source of power for PRU (70), with a battery (96) being used as a backup power source. Alternatively, battery (96) may be used as a primary source of power for PRU, with external power source (102) being used for backup power and/or to charge battery (96). External monitor (104) may be used to supplement or to substitute the display features of touch screen assembly (42) and/or touch screen assembly (72). For instance, external monitor (104) may display information including patient physiological parameters, status of operation of system (10), warning alerts, etc. PRU (70) and/or BMU (40) may communicate with external monitor (104) via cable, wirelessly (e.g., via RF transmission, etc.), or otherwise. Other examples of components, features, and functionality that may be incorporated into PRU (70) will be described in greater detail below; while still further examples of components, features, and functionality that may be incorporated into PRU (70) will be apparent to those of ordinary skill in the art in view of the teachings herein.

II. Exemplary System and Method to Manage Sedation Continuum

Those of ordinary skill in the art will recognize that a given patient's state of sedation may fall somewhere within a continuum of sedation, such that there are various degrees to which the patient may be sedated. Those of ordinary skill in the art will also recognize that it may be desirable to determine where exactly the patient's level of sedation falls within the continuum, with some degree of precision. As noted above, some conventional sedation systems may provide limited information regarding the level of sedation of a patient. For instance, a conventional ARM system may rely on audible and/or tactile responses from the patient in response to audible and/or tactile stimulus to indicate consciousness. Such an ARM system may simply indicate whether the patient has transitioned from a state of non-sedation or a state of minimum sedation to a state of moderate sedation. However, the spectrum of sedation includes many further levels beyond the state of moderate sedation, and the conventional ARM system may be incapable of providing any information on where the patient's current level of sedation is after the patient has passed the state of moderate sedation. It may therefore be desirable to provide a system that provides a greater degree of precision in indicating a patient's level of sedation. By increasing the precision of a level of sedation determination, a sedation system may provide greater precision in targeting a certain level of sedation. Moreover, by increasing the precision of a level of sedation determination, a sedation system may provide greater precision in making real-time adjustments to achieve and maintain such a precisely targeted level of sedation.

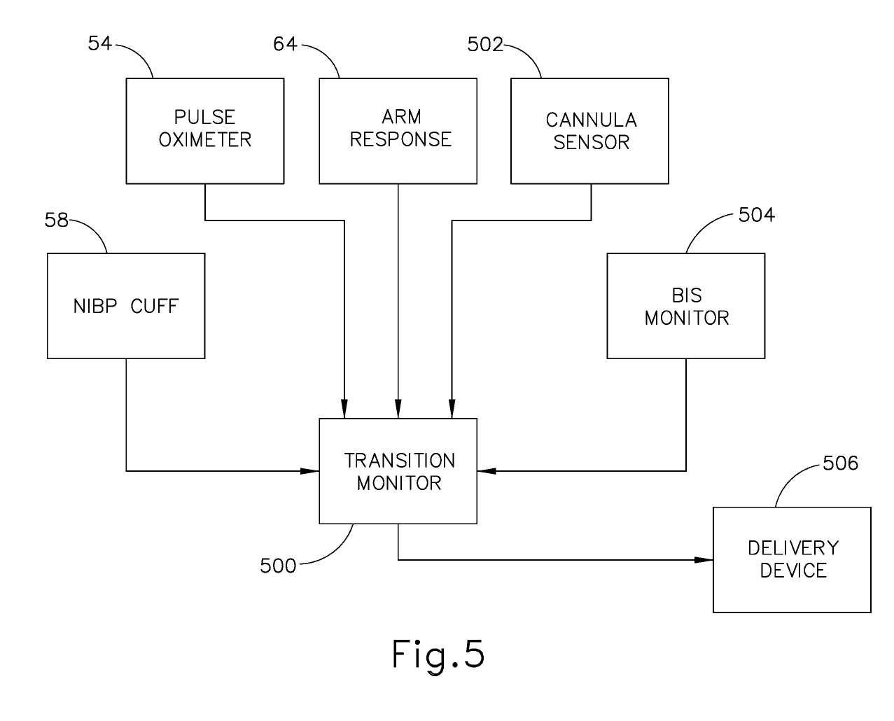

FIG. 5 depicts a schematic view of an exemplary set of devices providing data to a transition monitor (500) that is configured to manage sedation monitoring throughout the sedation continuum. Transition monitor (500) may comprise a device or software component and may be a standalone device or a device or software component that is incorporated into one or more other devices such as PRU (70) or BMU (40). Devices in communication with transition monitor (500) may include a variety of sensors and tools, including but not limited to an NIBP cuff (58), a pulse oximeter module (54), an ARM response module (64), a cannula sensor (502) that may detect the volume and contents of gases inhaled and exhaled by the patient, a bispectral index monitor (504), and/or other sensors or tools that may be used to provide information that may be indicative of a patient's level of sedation. Other suitable sensors or tools such as a basic EEG, manual patient assessment, and auditory evoked potentials, that may provide information that may be indicative of a patient's level of sedation and that may be coupled with transition monitor (500) will be apparent to those of ordinary skill in the art in view of the teachings herein. All of these various examples are contemplated as being included in the meaning of the term “sensor” and “sensors” as used herein.

Data provided by connected devices (54, 58, 64, 502, 504) may be provided to transition monitor (500) and other systems in parallel; or may be first provided to transition monitor (500), which may then provide the data to subsequent systems. Depending upon a particular implementation, transition monitor (500) may receive raw sensor data from connected devices (54, 58, 64, 502, 504), which it may be configured to interpret; or may receive pre-processed sensor data from connected devices (54, 58, 64, 502, 504) that it may use with minimal additional processing. Transition monitor (500) may additionally have the ability to display information, create audible alerts, and create visual alerts, with such abilities being made possible through devices integrated with transition monitor (500) or integrated with a device that is attached to transition monitor (500) such as PRU (70) or BMU (40). Thus, while PRU (70) and BMU (40) are not shown in FIG. 5, it should be understood that transition monitor (500) may be coupled with (or incorporated into) PRU (70) and/or BMU (40).

Transition monitor (500) may additionally be in communication with one or more delivery devices (506) which may include a drug delivery pump (84), cannula (94) oxygen supply, or similar delivery device. In some versions, delivery device (506) simply comprises PRU (70) as described above. Transition monitor (500) may be configured to start, stop, or adjust the delivery of drugs, oxygen, and/or other substances to a patient based upon data received through the sensors and devices that transition monitor (500) is in communication with. In other words, transition monitor (500) may be configured to make changes in the delivery of drugs, oxygen, and/or other substances to a patient, based on the level of sedation of the patient as sensed in real time.

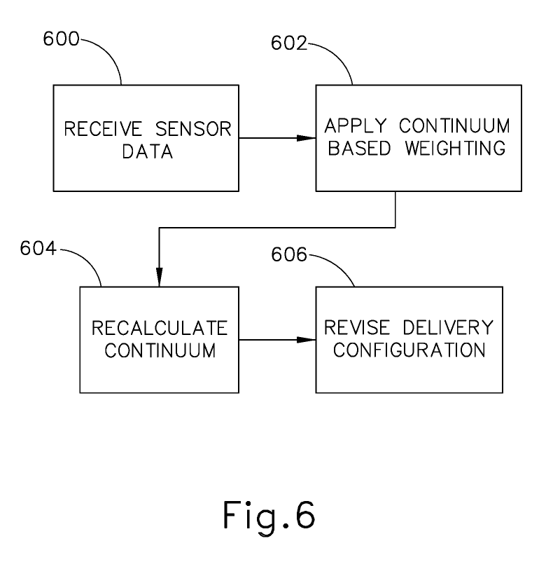

FIG. 6 depicts a high level flowchart of exemplary steps that may be performed by transition monitor (500) to maintain a sedation continuum over a period of time. As sensors generate data, the data is received (block 600) by transition monitor (500). A continuum based weighting is applied (block 602) to data as it is received. The weighting that is applied to each data point is dependent upon the source of the data point, such as the particular device, sensor, or tool from which the data originated; as well as the current zone of the sedation continuum that the system is operating within, such as minimum sedation, moderate sedation, or deep sedation. The sedation level may then be recalculated (block 604) based upon the newly received data (block 600) and weighted data (block 602). As the current sedation level is recalculated (block 604), a sedation level shifts or other changes may result in a delivery configuration being revised (block 606) for one or more attached delivery devices (506). For instance, delivery devices may deliver more drugs to the patient, deliver less drugs to the patient, deliver more or less oxygen to the patient, etc., based on the recalculated sedation level (block 604). The steps shown in FIG. 6 may be performed on multiple pieces of incoming data in parallel, or upon singular pieces or batches of data serially.

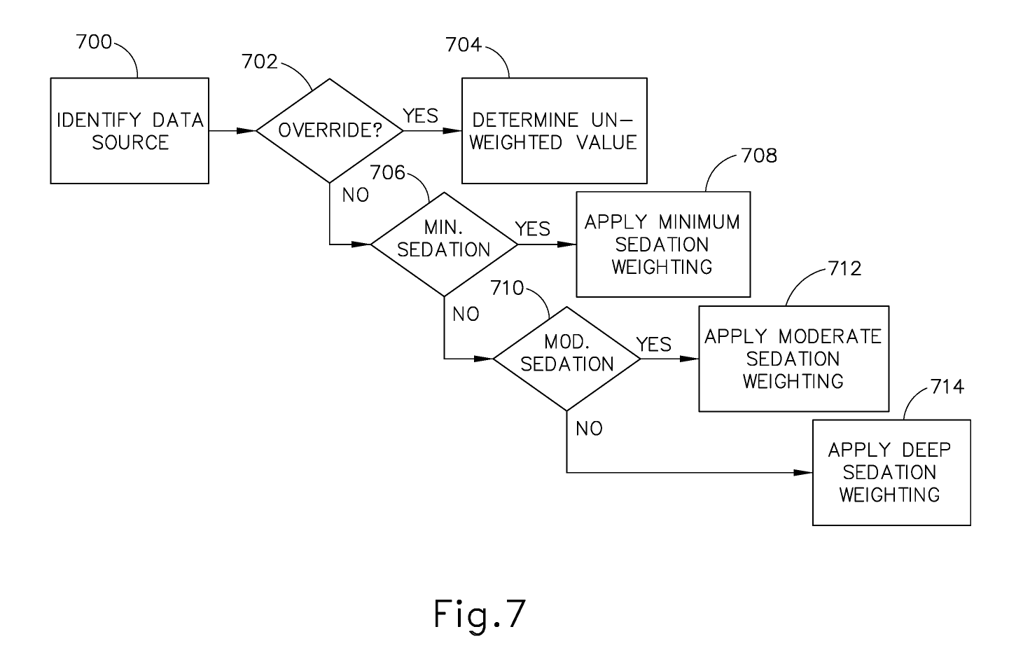

FIG. 7 depicts a flowchart of exemplary steps that may performed by transition monitor (500) to apply weighting to data received from a sensor (e.g., from connected devices (54, 58, 64, 502, 504) described above and/or from other devices). As data is received, the source of the data is identified (block 700). For example, data originating from an ARM response module (64) will be identified as being distinct from data originating from a pulse oximeter (54). Depending on the source of the data, it may be desirable to consider the data without determining a weighted value for the data. For example, it may be desirable to consider data originating from an ARM response module (64) without determining a weighted value for it, since a response received via the ARM response (64) is likely to have been caused by a conscious response from a patient. Therefore, it may not make sense to determine a weighted value for ARM responses during a deep sedation stage of the continuum when the ARM response itself likely indicates that the patient is not in a deep sedation stage of the continuum.

If there is an override (block 702) set for a certain data source within a stage of the continuum, the value of the data may be determined without applying any weighting (block 704). If there is no override (block 702), transition monitor (500) will determine what sedation stage the system is currently in and then apply the appropriate weighting to the data for that stage and data source. If the system is in a minimum sedation stage (block 706), a minimum sedation weighting may be applied (block 708). If the system is in a moderate sedation stage (block 710), a moderate sedation weighting may be applied (block 712). If the system is in a deep sedation stage, a deep sedation weighting may be applied (block 714).

As a merely illustrative example of this being carried out in practice, a patient may be undergoing a medical procedure where the patient is to be sedated. During this procedure three devices may provide data indicative of the patient's level of sedation to the transition monitor (500). The three devices in this example are an ARM response module (64), a BIS monitor (504), and a cannula sensor (502). The patient's level of sedation may be measured along the continuum by determining a total sedation value, with total sedation value between 1 and 33 indicating minimum sedation, total sedation between 34 and 66 indicating moderate sedation, and total sedation between 67 and 99 indicating deep sedation (e.g., with a level of 100 indicating that the patient is brain dead). The ARM response module (64) may be considered highly indicative of consciousness in at least some stages of the continuum (e.g., within the sedation values of 1 to 50). Because of this, ARM responses may have a weighted value of −90 for an ARM response to a verbal stimuli or a weighted value of −40 for an ARM response to a tactile stimulation while in a minimal sedation stage (i.e. −90/−40), −80/−35 in a moderate sedation stage, or −60/−25 in deep sedation stage. In this manner, ARM responses will reduce the patient's determined position along the sedation continuum by varying levels in the different stages of the continuum.

BIS monitor (504) may supply data indicative of consciousness level, but due to visual and auditory stimulation especially during minimal sedation the data may have unnecessary noise and, as a result, may not be reliable. Accordingly, monitor (504) data may have a weighting that, when applied to received data, results in a maximum value of 35 in minimum sedation, 50 in moderate sedation, and 100 in deep sedation. In this manner, BIS monitor (504) data is considered more reliable as the patient transitions into deep levels of sedation.

Cannula sensor (502) may supply data indicative of a patient's respiratory level during sedation, and may be considered reliable during minimal and moderate sedation. However, cannula sensor (502) may be considered unreliable during deep sedation, relative to data provided by BIS monitor (504) or another device. Accordingly, data from cannula sensor (502) may have a weighting that, when applied to received data, may result in a maximum value of 40 in minimal sedation, 40 in moderate sedation, and 20 in deep sedation.

Using these exemplary values and weights, a patient may begin a procedure at a minimal state of sedation. Verbal responses through ARM response module (64) will be given a high weight, and may keep the patient in a minimal sedation stage of the continuum regardless of the data received from BIS monitor (504) and cannula sensor (502), since the maximum possible value while receiving verbal ARM responses while in minimal sedation is 35+40−90=less than zero. When verbal responses through ARM response module (64) cease, indicating that the patient is transitioning to a moderate sedation stage, and the patient is only able to provide tactile responses through ARM response module (64), the maximum possible value then becomes 35+40−40=35, which may result in the transition monitor (500) transitioning to a moderate sedation monitoring configuration.

With transition monitor (500) in the the moderate sedation monitoring configuration, a verbal response through ARM response module (64) may still likely move the system back to a minimal sedation stage, due to its high relevance at all stages, but tactile responses through ARM response module (64) may occur while the system remains in the moderate sedation stage. When tactile responses through ARM response module (64) cease, indicating the patient is moving into deep sedation, the maximum possible value is 50+40 −0=90, sufficient to move the system into a deep sedation stage. While in deep sedation, an verbal or tactile responses through ARM response module (64) may not be enough to transition the system directly back to minimal sedation, but they are still given enough weight to transition the system back to a moderate continuum, in which continuing responses through ARM response module (64) may transition the system to a minimal sedation continuum. It should be appreciated that the values used in the example above are exemplary only, and the actual continuum scale used, the number of levels of sedation, and the weighted maximum or minimum values of various data sources in various stages of the continuum will vary greatly depending upon a particular embodiment and implementation, and may be reconfigured and changed over time by users in response to their uses and experiences with the system as well as in response to continuing medical study.

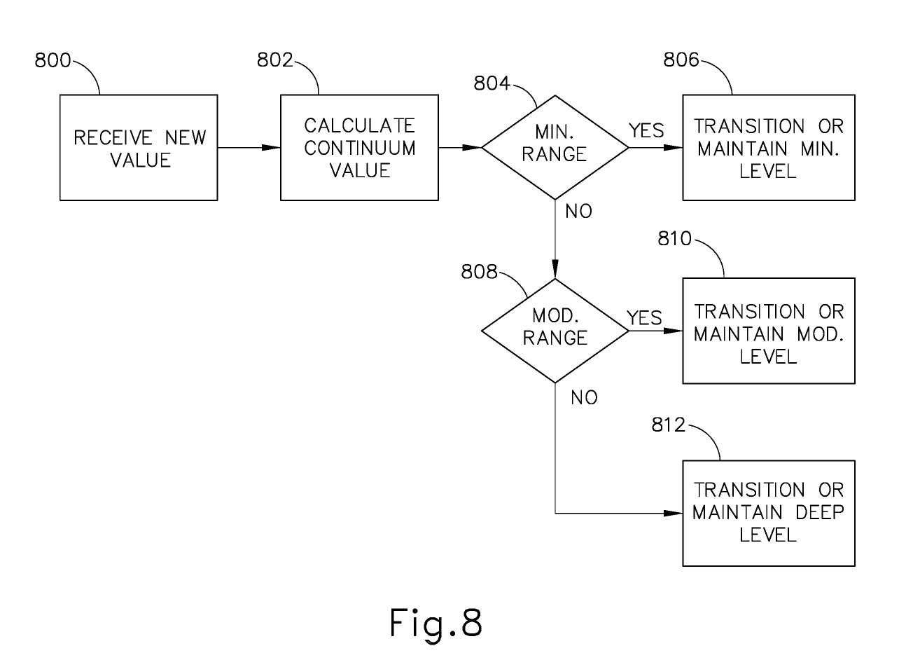

FIG. 8 depicts a flowchart of exemplary steps that may be performed by transition monitor (500) to recalculate and maintain a sedation level in response to data that has been received from one or more sensors; and to determine a weighted or un-weighted sedation continuum value. As weighted and un-weighted values are received (block 800) they may be used to calculate (block 802) a current sedation continuum value. This calculation may be a simple addition of multiple weighted values, as described above in the example of FIG. 7; or may be an ongoing calculation based upon a number of weighted values, where each additional value that arrives is added or subtracted from an existing sedatopm continuum value. For example, suppose a continuum value starts at zero, where each received value may increase or decrease the continuum value by 1-2 points, and where the continuum may shift from a minimal stage to a moderate stage when it exceeds 50 points. In this manner, rather than changing the sedation stage in real time based upon just a few of values as described above with reference to FIG. 7, the sedation stage would change only in response to a number of received values, from various sensors, over a period of time, with the period of time varying based upon the speed at which sensors provide data and the system processes data.

Regardless of the exact method used to calculate the sedation continuum value (block 802), once the sedation continuum value is available, transition monitor (500) will determine if the continuum value falls within a minimal sedation range (block 804), a moderate sedation range (block 808), or a deep sedation range. When a minimal sedation range (block 804) is detected, the system may transition to or maintain a minimal sedation configuration (block 806). When a moderate sedation range (block 808) is detected, the system may transition to or maintain a moderate sedation configuration (block 810). When a deep sedation range is detected, the system may transition to or maintain a deep sedation configuration (block 812). The exact ranges representing a minimal, moderate, or deep sedation range may vary depending upon the particular data available and weighting method applied, and the examples given above are exemplary only of a possible range.

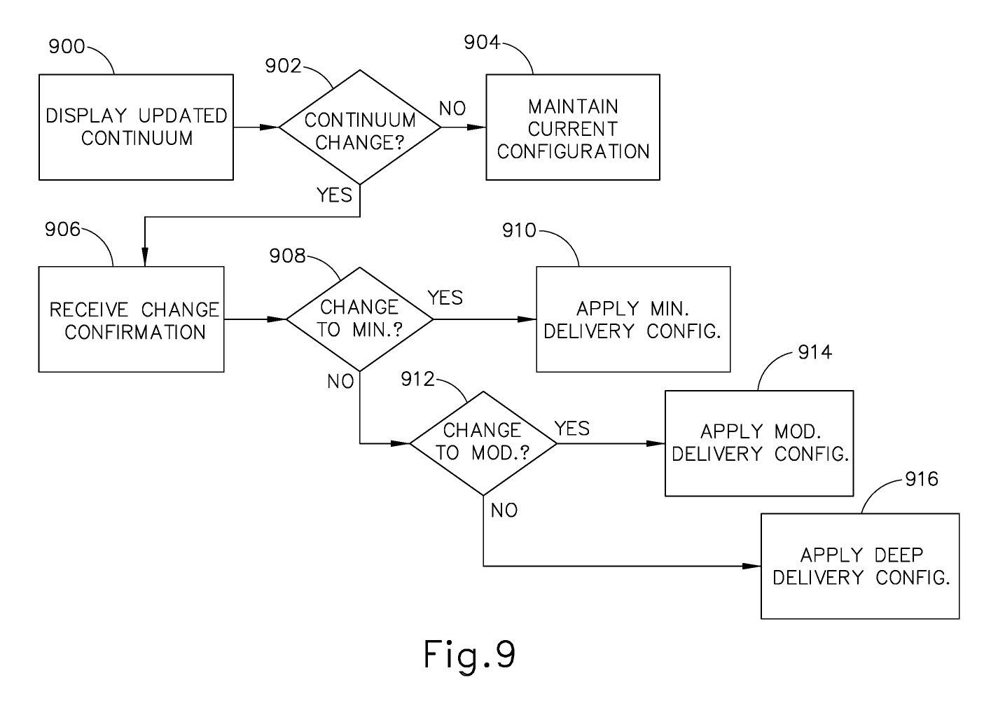

FIG. 9 depicts a flowchart of exemplary steps that may be performed using transition monitor (500) to transition drug delivery configurations from a first sedation state on the continuum to a second sedation state on the continuum. As a new continuum value is determined, the updated continuum may be displayed (block 900) via a display of the transition monitor (500); or via a display of the PRU (70), BMU (40), or other available display. If the new continuum value does not result in a continuum stage change, one or more drugs, oxygen, and/or other substance delivery may be maintained at its current configuration. If the new continuum value is determined to have resulted in a continuum change from one stage to another (block 806, block 810, block 812), the system may display a notification explaining that the transition along the sedation continuum has occurred and display information about the impact that the transition may have upon substance delivery (e.g., drug(s), oxygen, etc.) to the patient, which a clinician may review confirm via a display or control proximate to the system. The change confirmation may be received by the system (block 906) and will result in the system changing the substance delivery configurations to a configuration matching the new sedation continuum.

For example, when the sedation continuum transitions to a minimal stage (block 908), a minimal stage substance delivery configuration may be performed (block 910). When the sedation continuum transitions to a moderate stage (block 912), a moderate stage substance delivery configuration may be performed (block 914). When the sedation continuum transitions to a deep stage, a deep stage substance delivery configuration may be performed (block 916). Automatic management of substance delivery based upon the determined sedation continuum stage may cause one or more delivery devices (block 506) to start, stop, or vary the delivery of a drug, a respiratory mix, or another deliverable in a way that will help in maintaining the patient in the desired stage of the sedation continuum and prevent injury. In this manner, as a patient transitions from a moderate sedation into a deep sedation, the transition may cause the system to apply a deep sedation delivery configuration, which may result in a decrease of drug delivery for sedatives, as the patient has reached a desired deep sedation level, and may begin delivery of concentrated oxygen to help the patient maintain a healthy oxygen level during a period of reduced respiratory activity.

The particular delivery configurations for minimum, moderate, and deep sedation may vary by example and implementation; and may also vary by factors such as patient physiology and history, procedure being performed, equipment being used in the procedure, and other similar factors. It should also be understood the breakdown of the sedation continuum into three zones (minimum, moderate, and deep) in the present example is merely one example. The sedation continuum may alternatively be broken into any other suitable number of zones, including more or less than three.

III. Exemplary Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### EXAMPLE 1

An apparatus comprising: (a) a sedation transition monitor, the sedation transition monitor comprising a memory, wherein the memory is configured to store a continuum stage indicator; and (b) a set of sensors, wherein the set of sensors is communicatively coupled with the sedation transition monitor, the set of sensors comprising a first sensor; wherein the sedation transition monitor is configured to: (i) receive a set of sensor data from the set of sensors, wherein the set of sensor data comprises a first sensor data, (ii) identify the first sensor that the first sensor data originated from, (iii) determine a sedation stage value for the first sensor data using a first data weighting calculation, wherein the first data weighting calculation is selected by the sedation transition monitor based upon the identity of the first sensor and the continuum stage indicator, (iv) determine a new continuum value based at least in part upon the sedation stage value, and (v) update the continuum stage indicator to reflect the new continuum value.

### EXAMPLE 2

The apparatus of Example 1, wherein the set of sensors further comprises a second sensor, wherein the set of sensor data further comprises a second sensor data, and wherein the sedation transition monitor is further configured to: (i) identify the second sensor that the second sensor data originated from, (ii) determine the sedation stage value for the second sensor data using a second data weighting calculation, wherein sedation transition monitor is configured to select the second data weighting calculation based upon the identity of the second sensor and the continuum stage indicator, (iii) determine the new continuum value based at least in part upon the sedation stage value, and (iv) update the continuum stage indicator to reflect the new continuum value; wherein the first data weighting calculation and the second data weighting calculation are not identical.

### EXAMPLE 3

The apparatus of Example 2, wherein the first sensor comprises an automated responsiveness monitor, and wherein the second sensor comprises a bispectral index monitor, wherein the first data weighting calculation is configured to result in a higher sedation stage value for the first sensor data when the continuum stage indicator indicates a low sedation stage, as compared to when the continuum stage indicator indicates a deep sedation stage; and wherein the second data weighting calculation is configured to result in a higher sedation stage value for the second sensor data when the continuum stage indicator indicates the deep sedation stage, as compared to when the continuum stage indicator indicates the low sedation stage.

### EXAMPLE 4

The apparatus of any one or more of Examples 1 through 3, wherein the sedation transition monitor is further configured to, in response to the first sensor being associated with an override indicator, determine the sedation stage value for the first sensor data using an override data weighting calculation, wherein the override data weighting calculation is selected by the sedation transition monitor based upon the identity of the first sensor.

### EXAMPLE 5

The apparatus of any one or more of Examples 1 through 4, wherein the continuum stage indicator is capable of representing, singularly and in response to being updated by the sedation transition monitor, sedation stages consisting of a minimal sedation stage, a moderate sedation stage, a deep sedation stage, and a general anesthesia sedation stage.

### EXAMPLE 6

The apparatus of any one or more of Examples 1 through 5, wherein the sedation transition monitor is further configured to determine the new continuum value based at least in part upon the sedation stage value and a previous continuum value.

### EXAMPLE 7

The apparatus of any one or more of Examples 1 through 6, wherein the sedation transition monitor comprises one or more of a bedside monitor unit and a procedure room unit.

### EXAMPLE 8

The apparatus of any one or more of Examples 1 through 7, further comprising a substance delivery device in communication with the sedation transition monitor, wherein the substance delivery device is operable to deliver one or more substances to a patient, wherein the transitional monitor is further configured to adjust a delivery rate of the substance delivery device in response to an update to the continuum stage indicator.

### EXAMPLE 9

The apparatus of Example 8, wherein the substance delivery device comprises one or more of a drug delivery pump, a drug delivery cassette, or an oxygen delivery source.

### EXAMPLE 10

The apparatus of any one or more of Examples 1 through 9, wherein the set of sensors comprises a blood pressure monitor, a pulse oximeter, an automated responsiveness monitor, a cannula sensor, and a bispectral index monitor.

### EXAMPLE 11

A method comprising: (a) storing a continuum stage indicator in a memory of a sedation transition monitor; (b) receiving, at the sedation transition monitor, a set of sensor data from a set of sensors communicatively coupled with the sedation transition monitor, wherein the set of sensors are configured to monitor one or more biological properties of a patient and thereby collect data indicative of a sedation state of the patient, the set of sensors comprising a first sensor, the set of sensor data comprising a first sensor data; (c) identifying, at the sedation transition monitor, the first sensor that the first sensor data originated from; (d) selecting, based upon the first sensor and the continuum stage indicator, a first data weighting calculation; (e) determining a sedation stage value for the first sensor data using the first data weighting calculation; (f) determining a new continuum value based at least in part upon the sedation stage value; and (g) updating the continuum stage indicator to reflect the new continuum value.

### EXAMPLE 12

The method of Example 11, wherein the set of sensors further comprises a second sensor, wherein the set of sensor data further comprises a second sensor data, the method further comprising: (a) identifying the second sensor that the second sensor data originated from; (b) selecting a second data weighting calculation based upon the second sensor and the continuum stage indicator; (c) determining the sedation stage value for the second sensor data using the second data weighting calculation; (d) determining the new continuum value based at least in part upon the sedation stage value; and (e) updating the continuum stage indicator to reflect the new continuum value; wherein the first data weighting calculation and the second data weighting calculation are not identical.

### EXAMPLE 13

The method of Example 12, wherein the first sensor comprises an automated responsiveness monitor configured to receive responses from the patient in response to stimulus, and wherein the second sensor comprises a bispectral index monitor configured to sense electrical activity of the patient's brain, the method further comprising: (a) configuring the first data weighting calculation to result in a higher sedation stage value for the first sensor data when the continuum stage indicator indicates a low sedation stage, as compared to when the continuum stage indicator indicates a deep sedation stage; and (b) configuring the second data weighting calculation to result in a higher sedation stage value for the second sensor data when the continuum stage indicator indicates the deep sedation stage, as compared to when the continuum stage indicator indicates the low sedation stage.

### EXAMPLE 14

The method of any one or more of Examples 11 through 13, further comprising the step of, in response to the first sensor being associated with an override indicator: (a) selecting, at the sedation transition monitor, an override data weighting calculation based upon the first sensor; and (a) determining the sedation stage value for the first sensor data using only the override data weighting calculation.

### EXAMPLE 15

The method of any one or more of Examples 11 through 14, further comprising the step of configuring the continuum stage indicator to be capable of storing, singularly and in response to being updated by the sedation transition monitor, sedation stages consisting of a minimal sedation stage, a moderate sedation stage, a deep sedation stage, and a general anesthesia sedation stage.

### EXAMPLE 16

The method of any one or more of Examples 11 through 15, wherein determining the new continuum value based at least in part upon the sedation stage value further comprises the step determining the new continuum value based at least in part upon the sedation stage value and a previous continuum value.

### EXAMPLE 17

The method of any one or more of Examples 11 through 16, further comprising:

(a) configuring the sedation transition monitor to communicate with a substance delivery device, wherein the substance delivery device delivers a substance to a patient; and (b) adjusting a rate at which the substance delivery device delivers the substance to the patient, wherein the act of adjusting is performed in response to updating the continuum stage indicator.

### EXAMPLE 18

The method of Example 17, wherein the substance delivery device comprises one or more of a drug delivery pump, a drug delivery cassette, or an oxygen delivery source.

### EXAMPLE 19

The method of any one or more of Examples 11 through 18, wherein the first sensor comprises an automated responsiveness monitor configured to receive responses from the patient in response to stimulus, the method further comprising: (a) receiving a response via the automated responsiveness monitor; and (b) in response to receiving the response via the automated responsiveness monitor, updating the continuum stage indicator to reflect a high level of consciousness.

### EXAMPLE 20

An apparatus comprising: (a) a sedation transition monitor, the sedation transition monitor comprising a memory, wherein the memory is configured to store a continuum stage indicator, wherein the continuum stage indicator is capable of representing, singularly, sedation stages consisting of a minimal sedation stage, a moderate sedation stage, a deep sedation stage, and a general anesthesia sedation stage; (b) a set of sensors, wherein the set of sensors are communicatively coupled with the sedation transition monitor, the set of sensors comprising a first sensor and a second sensor; and (c) a drug delivery device; wherein the sedation transition monitor is configured to: (i) receive a set of sensor data from the set of sensors, the set of sensor data comprising a first sensor data and a second sensor data, (ii) identify the first sensor that the first sensor data originated from, wherein the first sensor is an automated responsiveness monitor, (iii) determine a sedation stage value for the first sensor data using a first data weighting calculation selected by the sedation transition monitor based upon the identity of the first sensor and the continuum stage indicator, and wherein the first data weighting calculation is configured to result in a higher sedation stage value for the first sensor data when the continuum stage indicator indicates a low sedation stage, as compared to when the continuum stage indicator indicates a deep sedation stage, (iv) determine a new continuum value based at least in part upon the sedation stage value, (v) update the continuum stage indicator to reflect the new continuum value and adjust a delivery rate of the drug delivery device based upon the continuum stage indicator, (vi) identify the second sensor that the second sensor data originated from, wherein the second sensor is a bispectral index monitor, (vii) determine the sedation stage value for the second sensor data using a second data weighting calculation, wherein the second data weighting calculation is selected by the sedation transition monitor based upon the identity of the second sensor and the continuum stage indicator, wherein the second data weighting calculation is configured to result in a higher sedation stage value for the second sensor data when the continuum stage indicator indicates the deep sedation stage, as compared to when the continuum stage indicator indicates the low sedation stage; (viii) determine the new continuum value based at least in part upon the sedation stage value, and (ix) update the continuum stage indicator to reflect the new continuum value and adjust the delivery rate of the drug delivery device based upon the continuum stage indicator; wherein the first data weighting calculation and the second data weighting calculation are not identical.

IV. Miscellaneous

It should be understood that any of the examples described herein may include various other features in addition to or in lieu of those described above. By way of example only, any of the devices herein may also include one or more of the various features disclosed in any of the various references that are incorporated by reference herein. It should also be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

It should also be understood that any ranges of values referred to herein should be read to include the upper and lower boundaries of such ranges. For instance, a range expressed as ranging “between approximately 1.0 inches and approximately 1.5 inches” should be read to include approximately 1.0 inches and approximately 1.5 inches, in addition to including the values between those upper and lower boundaries.

Versions of the devices described above may have application in conventional medical treatments and procedures conducted by a medical professional, as well as application in robotic-assisted medical treatments and procedures. By way of example only, various teachings herein may be readily incorporated into a robotic surgical system such as the DAVINCI™ system by Intuitive Surgical, Inc., of Sunnyvale, Calif.

Similarly, those of ordinary skill in the art will recognize that various teachings herein may be readily combined with various teachings of U.S. Pat. No. 6,783,524, entitled “Robotic Surgical Tool with Ultrasound Cauterizing and Cutting Instrument,” published Aug. 31, 2004, the disclosure of which is incorporated by reference herein.

Versions described above may be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, some versions of the device may be disassembled, and any number of the particular pieces or parts of the device may be selectively replaced or removed in any combination. Upon cleaning and/or replacement of particular parts, some versions of the device may be reassembled for subsequent use either at a reconditioning facility, or by an operator immediately prior to a procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be sterilized before and/or after a procedure. In one sterilization technique, the device is placed in a closed and sealed container, such as a plastic or TYVEK bag. The container and device may then be placed in a field of radiation that can penetrate the container, such as gamma radiation, x-rays, or high-energy electrons. The radiation may kill bacteria on the device and in the container. The sterilized device may then be stored in the sterile container for later use. A device may also be sterilized using any other technique known in the art, including but not limited to beta or gamma radiation, ethylene oxide, or steam.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. An apparatus comprising:
(a) a sedation transition monitor, the sedation transition monitor comprising a memory, wherein the memory is configured to store a continuum stage indicator; and
(b) a set of sensors, wherein the set of sensors is communicatively coupled with the sedation transition monitor, the set of sensors comprising a first sensor;
wherein the sedation transition monitor is configured to:
(i) receive a set of sensor data from the set of sensors, wherein the set of sensor data comprises a first sensor data,
(ii) identify the first sensor that the first sensor data originated from,
(iii) determine a sedation stage value for the first sensor data using a first data weighting calculation, wherein the first data weighting calculation is selected by the sedation transition monitor based upon the identity of the first sensor and the continuum stage indicator,
(iv) determine a new continuum value based at least in part upon the sedation stage value, and
(v) update the continuum stage indicator to reflect the new continuum value.

2. The apparatus of claim 1, wherein the set of sensors further comprises a second sensor, wherein the set of sensor data further comprises a second sensor data, and wherein the sedation transition monitor is further configured to:
(i) identify the second sensor that the second sensor data originated from,
(ii) determine the sedation stage value for the second sensor data using a second data weighting calculation, wherein sedation transition monitor is configured to select the second data weighting calculation based upon the identity of the second sensor and the continuum stage indicator,
(iii) determine the new continuum value based at least in part upon the sedation stage value, and
(iv) update the continuum stage indicator to reflect the new continuum value;
wherein the first data weighting calculation and the second data weighting calculation are not identical.

3. The apparatus of claim 2, wherein the first sensor comprises an automated responsiveness monitor, and wherein the second sensor comprises a bispectral index monitor, wherein the first data weighting calculation is configured to result in a higher sedation stage value for the first sensor data when the continuum stage indicator indicates a low sedation stage, as compared to when the continuum stage indicator indicates a deep sedation stage; and
wherein the second data weighting calculation is configured to result in a higher sedation stage value for the second sensor data when the continuum stage indicator indicates the deep sedation stage, as compared to when the continuum stage indicator indicates the low sedation stage.

4. The apparatus of claim 1, wherein the sedation transition monitor is further configured to, in response to the first sensor being associated with an override indicator, determine the sedation stage value for the first sensor data using an override data weighting calculation, wherein the override data weighting calculation is selected by the sedation transition monitor based upon the identity of the first sensor.

5. The apparatus of claim 1, wherein the continuum stage indicator is capable of representing, singularly and in response to being updated by the sedation transition monitor, sedation stages consisting of a minimal sedation stage, a moderate sedation stage, a deep sedation stage, and a general anesthesia sedation stage.

6. The apparatus of claim 1, wherein the sedation transition monitor is further configured to determine the new continuum value based at least in part upon the sedation stage value and a previous continuum value.

7. The apparatus of claim 1, wherein the sedation transition monitor comprises one or more of a bedside monitor unit and a procedure room unit.

8. The apparatus of claim 1, further comprising a substance delivery device in communication with the sedation transition monitor, wherein the substance delivery device is operable to deliver one or more substances to a patient, wherein the transitional monitor is further configured to adjust a delivery rate of the substance delivery device in response to an update to the continuum stage indicator.

9. The apparatus of claim 8, wherein the substance delivery device comprises one or more of a drug delivery pump, a drug delivery cassette, or an oxygen delivery source.

10. The apparatus of claim 1, wherein the set of sensors comprises a blood pressure monitor, a pulse oximeter, an automated responsiveness monitor, a cannula sensor, and a bispectral index monitor.

11. A method comprising:
(a) storing a continuum stage indicator in a memory of a sedation transition monitor;
(b) receiving, at the sedation transition monitor, a set of sensor data from a set of sensors communicatively coupled with the sedation transition monitor, wherein the set of sensors are configured to monitor one or more biological properties of a patient and thereby collect data indicative of a sedation state of the patient, the set of sensors comprising a first sensor, the set of sensor data comprising a first sensor data;
(c) identifying, at the sedation transition monitor, the first sensor that the first sensor data originated from;
(d) selecting, based upon the first sensor and the continuum stage indicator, a first data weighting calculation;
(e) determining a sedation stage value for the first sensor data using the first data weighting calculation;
(f) determining a new continuum value based at least in part upon the sedation stage value; and
(g) updating the continuum stage indicator to reflect the new continuum value.

12. The method of claim 11, wherein the set of sensors further comprises a second sensor, wherein the set of sensor data further comprises a second sensor data, the method further comprising:
(a) identifying the second sensor that the second sensor data originated from;
(b) selecting a second data weighting calculation based upon the second sensor and the continuum stage indicator;
(c) determining the sedation stage value for the second sensor data using the second data weighting calculation;
(d) determining the new continuum value based at least in part upon the sedation stage value; and
(e) updating the continuum stage indicator to reflect the new continuum value;
wherein the first data weighting calculation and the second data weighting calculation are not identical.

13. The method of claim 12, wherein the first sensor comprises an automated responsiveness monitor configured to receive responses from the patient in response to stimulus, and wherein the second sensor comprises a bispectral index monitor configured to sense electrical activity of the patient's brain, the method further comprising:
(a) configuring the first data weighting calculation to result in a higher sedation stage value for the first sensor data when the continuum stage indicator indicates a low sedation stage, as compared to when the continuum stage indicator indicates a deep sedation stage; and
(b) configuring the second data weighting calculation to result in a higher sedation stage value for the second sensor data when the continuum stage indicator indicates the deep sedation stage, as compared to when the continuum stage indicator indicates the low sedation stage.

14. The method of claim 11, further comprising the step of, in response to the first sensor being associated with an override indicator:
(a) selecting, at the sedation transition monitor, an override data weighting calculation based upon the first sensor; and
(a) determining the sedation stage value for the first sensor data using only the override data weighting calculation.

15. The method of claim 11, further comprising the step of configuring the continuum stage indicator to be capable of storing, singularly and in response to being updated by the sedation transition monitor, sedation stages consisting of a minimal sedation stage, a moderate sedation stage, a deep sedation stage, and a general anesthesia sedation stage.

16. The method of claim 11, wherein determining the new continuum value based at least in part upon the sedation stage value further comprises the step determining the new continuum value based at least in part upon the sedation stage value and a previous continuum value.

17. The method of claim 11, further comprising:
(a) configuring the sedation transition monitor to communicate with a substance delivery device, wherein the substance delivery device delivers a substance to a patient; and
(b) adjusting a rate at which the substance delivery device delivers the substance to the patient, wherein the act of adjusting is performed in response to updating the continuum stage indicator.

18. The method of claim 17, wherein the substance delivery device comprises one or more of a drug delivery pump, a drug delivery cassette, or an oxygen delivery source.

19. The method of claim 11, wherein the first sensor comprises an automated responsiveness monitor configured to receive responses from the patient in response to stimulus, the method further comprising:
(a) receiving a response via the automated responsiveness monitor; and
(b) in response to receiving the response via the automated responsiveness monitor, updating the continuum stage indicator to reflect a high level of consciousness.

20. An apparatus comprising:
(a) a sedation transition monitor, the sedation transition monitor comprising a memory, wherein the memory is configured to store a continuum stage indicator, wherein the continuum stage indicator is capable of representing, singularly, sedation stages consisting of a minimal sedation stage, a moderate sedation stage, a deep sedation stage, and a general anesthesia sedation stage;
(b) a set of sensors, wherein the set of sensors are communicatively coupled with the sedation transition monitor, the set of sensors comprising a first sensor and a second sensor; and
(c) a drug delivery device;
wherein the sedation transition monitor is configured to:
(i) receive a set of sensor data from the set of sensors, the set of sensor data comprising a first sensor data and a second sensor data,
(ii) identify the first sensor that the first sensor data originated from, wherein the first sensor is an automated responsiveness monitor,
(iii) determine a sedation stage value for the first sensor data using a first data weighting calculation selected by the sedation transition monitor based upon the identity of the first sensor and the continuum stage indicator, and wherein the first data weighting calculation is configured to result in a higher sedation stage value for the first sensor data when the continuum stage indicator indicates a low sedation stage, as compared to when the continuum stage indicator indicates a deep sedation stage,
(iv) determine a new continuum value based at least in part upon the sedation stage value,
(v) update the continuum stage indicator to reflect the new continuum value and adjust a delivery rate of the drug delivery device based upon the continuum stage indicator,
(vi) identify the second sensor that the second sensor data originated from, wherein the second sensor is a bispectral index monitor,
(vii) determine the sedation stage value for the second sensor data using a second data weighting calculation, wherein the second data weighting calculation is selected by the sedation transition monitor based upon the identity of the second sensor and the continuum stage indicator, wherein the second data weighting calculation is configured to result in a higher sedation stage value for the second sensor data when the continuum stage indicator indicates the deep sedation stage, as compared to when the continuum stage indicator indicates the low sedation stage;
(viii) determine the new continuum value based at least in part upon the sedation stage value, and
(ix) update the continuum stage indicator to reflect the new continuum value and adjust the delivery rate of the drug delivery device based upon the continuum stage indicator;
wherein the first data weighting calculation and the second data weighting calculation are not identical.
